SEQUENCE LISTING (41 sequences)

SEQ ID NO: 1 - DNA, 13 bp, Artificial Sequence
  SfiI site
ggccnnnnnggcc

SEQ ID NO: 2 - DNA, 26 bp, Artificial Sequence
  nLIC fd primer
atggtgagaatttatattttcaaggt

SEQ ID NO: 3 - DNA, 22 bp, Artificial Sequence
  nLIC rv primer
tgggagggtgggattttcatta

SEQ ID NO: 4 - DNA, 18 bp, Artificial Sequence
  cLIC fd primer
atgggtggtggatttgct

SEQ ID NO: 5 - DNA, 18 bp, Artificial Sequence
  cLIC rv primer
ttggaagtataaattttc

SEQ ID NO: 6 - DNA, 67 bp, Artificial Sequence
  nLIC  cassette
atgcatcatcaccatcatcaccatcaccatcatggtgagaatttatatttaaatcccaccctcccag

SEQ ID NO: 7 - protein, 16 aa, Artificial Sequence
  Synthetic Construct
MHHHHHHHHHHGENLY

SEQ ID NO: 8 - DNA, 50 bp, Artificial Sequence
  SwaI digestion product 1 of nLIC cassette
atgcatcatcaccatcatcaccatcaccatcatggtgagaatttatattt

SEQ ID NO: 9 - DNA, 17 bp, Artificial Sequence
  SwaI digestion product 2 of nLIC cassette
aaatcccaccctcccag

SEQ ID NO: 10 - DNA, 31 bp, Artificial Sequence
  nLIC after SwaI and T4 treatment
atgcatcatcaccatcatcaccatcaccatc

SEQ ID NO: 11 - protein, 10 aa, Artificial Sequence
  Synthetic Construct
MHHHHHHHHH

SEQ ID NO: 12 - DNA, 29 bp, Artificial Sequence
  nLIC PCR(1)
atggtgagaatttatattttcaaggtatg

SEQ ID NO: 13 - protein, 9 aa, Artificial Sequence
  Synthetic Construct
GENLYFQGM

SEQ ID NO: 14 - DNA, 22 bp, Artificial Sequence
  nLIC PCR(2)
taatgaaaatcccaccctccca

SEQ ID NO: 15 - DNA, 26 bp, Artificial Sequence
  PCR + T4 product (1)
atggtgagaatttatattttcaaggt

SEQ ID NO: 16 - protein, 8 aa, Artificial Sequence
  Synthetic Construct
GENLYFQG

SEQ ID NO: 17 - DNA, 22 bp, Artificial Sequence
  PCR + T4 product (2)
tgggagggtgggattttcatta

SEQ ID NO: 18 - DNA, 57 bp, Artificial Sequence
  nLIC 5′ end after annealing
atgcatcatcaccatcatcaccatcaccatcatggtgagaatttatattttcaaggt

SEQ ID NO: 19 - protein, 19 aa, Artificial Sequence
  Synthetic Construct
MHHHHHHHHHHGENLYFQG

SEQ ID NO: 20 - DNA, 17 bp, Artificial Sequence
  nLIC 3′ end after annealing (1)
aaatcccaccctcccag

SEQ ID NO: 21 - DNA, 23 bp, Artificial Sequence
  nLIC 3′ end after annealing (2)
ctgggagggtgggattttcatta

SEQ ID NO: 22 - DNA, 68 bp, Artificial Sequence
  cLIC cassette
catgggtggtggatttaaatttatacttccaaggtcatcatcaccatcatcaccatcaccatcattaa

SEQ ID NO: 23 - protein, 5 aa, Artificial Sequence
  Synthetic Construct
MGGGF

SEQ ID NO: 24 - protein, 16 aa, Artificial Sequence
  Synthetic Construct
NLYFQGHHHHHHHHHH

SEQ ID NO: 25 - DNA, 16 bp, Artificial Sequence
  cLIC after SwaI (1)
catgggtggtggattt

SEQ ID NO: 26 - DNA, 52 bp, Artificial Sequence
  cLIC after SwaI (2)
aaatttatacttccaaggtcatcatcaccatcatcaccatcaccatcattaa

SEQ ID NO: 27 - DNA, 36 bp, Artificial Sequence
  cLIC after SwaI + T4
ttaatgatggtgatggtgatgatggtgatgatgacc

SEQ ID NO: 28 - DNA, 18 bp, Artificial Sequence
  cLIC PCR(1)
atgggtggtggatttgct

SEQ ID NO: 29 - protein, 6 aa, Artificial Sequence
  Synthetic Construct
MGGGFA

SEQ ID NO: 30 - DNA, 18 bp, Artificial Sequence
  cLIC PCR(2)
gaaaatttatacttccaa

SEQ ID NO: 31 - protein, 6 aa, Artificial Sequence
  Synthetic Construct
ENLYFQ

SEQ ID NO: 32 - DNA, 19 bp, Artificial Sequence
  cLIC 5′ end after annealing (1)
catgggtggtggatttgct

SEQ ID NO: 33 - protein, 6 aa, Artificial Sequence
  Synthetic Construct
MGGGFA

SEQ ID NO: 34 - DNA, 16 bp, Artificial Sequence
  cLIC 5′ end after annealing (2)
catgggtggtggattt

SEQ ID NO: 35 - protein, 5 aa, Artificial Sequence
  Synthetic Construct
MGGGF

SEQ ID NO: 36 - DNA, 52 bp, Artificial Sequence; identical to SEQ ID NO: 26
  cLIC 3′ end after annealing (1)

SEQ ID NO: 37 - protein, 16 aa, Artificial Sequence
  Synthetic Construct
NLYFQGHHHHHHHHHH

SEQ ID NO: 38 - DNA, 54 bp, Artificial Sequence
  cLIC 3′ end after annealing (2)
gaaaatttatacttccaaggtcatcatcaccatcatcaccatcaccatcattaa

SEQ ID NO: 39 - protein, 17 aa, Artificial Sequence
  Synthetic Construct
ENLYFQGHHHHHHHHHH

SEQ ID NO: 40 - DNA, 13 bp, Artificial Sequence
  SfiI x
ggccactgaggcc

SEQ ID NO: 41 - DNA, 13 bp, Artificial Sequence
  SfiI y
ggccggacgggcc

(19) United States
(12) Patent Application Publication
(10) Pub. No.: US 2011/0033909 A1
(43) Pub. Date: Feb. 10, 2011

(54) MEANS AND METHODS FOR CLONING NUCLEIC ACID SEQUENCES

(75) Inventors: Eric Robin Geertsma, Groningen (NL); Berend Poolman, Haren (NL)

Correspondence Address: REED SMITH LLP, 2500 ONE LIBERTY PLACE, 1650 MARKET STREET, PHILADELPHIA, PA, 19103, US

(73) Assignee: RIJKSUNIVERSITEIT GRONINGEN, Groningen (NL)

(21) Appl. No.: 12/602,525

(22) PCT Filed: May 30, 2008
(86) PCT No.: PCT/NL2008/050329

§ 371 Date: Feb. 2, 2010

Related U.S. Application Data
(60) Provisional application No. 60/932,686, filed on Jun. 1, 2007.

(30) Foreign Application Priority Data
Jun. 1, 2007 (EP) 07109450.2

(51) Int. Cl. C12P 1/04; C12N 15/74

(52) U.S. Cl. 435/170; 435/471

(57) ABSTRACT

The invention provides means and methods for efficiently cloning nucleic acid sequences of interest in micro-organisms that are less amenable to conventional nucleic acid manipulations, as compared to, for instance, E.coli. The present invention enables high-throughput cloning (and, preferably, expressing) of a nucleic acid of interest in a first kind of micro-organism, while an initial (preferably high throughput) cloning of the nucleic acid of interest is done in a second kind of micro-organism.

The invention relates to the field of molecular biology.

Micro-organisms are an attractive host for amplifying and expressing nucleic acid. Expression of nucleic acids which encode protein is a valuable tool for producing sufficient amounts of a protein of interest, such as for instance (therapeutic) enzymes. Well-established expression hosts are Escherichia coli and yeast.

However, proteins produced this way do not always have the native conformation or they are not at all produced. For instance, proteins are not always folded correctly by the micro-organisms that produce them and may contain unwanted post-translational modifications. Heterologous expression of large multidomain assemblies and membrane proteins in a functionally competent state is often problematic in the well-established expression hosts E. coli and yeast.

Alternative expression systems are thus urgently needed to overcome this major hurdle in structural genomics projects. Frequently, efficient DNA manipulations form a bottleneck for exploring the protein expression potential of novel hosts, thereby preventing rapid and routine screening. Shuttle vectors that replicate both in E. coli and an alternative host offer a way out, but their use is complicated by the requirement for dual replication factors and multiple selection markers. This increases the plasmid size and often compromises stable propagation. For instance, Lactococcus lactis is widely recognized as an attractive alternative to E. coli and yeast-based expression systems. However, the expression screening in L. lactis has been seriously hampered by the low efficiency of gene manipulations in the organism and the instability of L. lactis-E. coli shuttle vectors. Additionally, the lack of an efficient cloning procedure has restricted the preparation of large gene libraries for directed evolution studies in L. lactis; similar limitations have prohibited the full exploitation of other micro-organisms as expression and screening hosts.

It is an object of the present invention to provide alternative means and methods for cloning a nucleic acid of interest in a micro-organism. Preferably, means and methods are provided for cloning a nucleic acid of interest in a micro-organism which is less amenable to conventional nucleic acid manipulations as compared to E.coli.

Accordingly, the present invention provides a method for cloning a nucleic acid sequence of interest in a first kind of micro-organism, the method comprising:

inserting said nucleic acid of interest into a nucleic acid vehicle, which vehicle comprises an origin of replication of a second kind of micro-organism;

cloning said vehicle in a culture of said second kind of micro-organism;

isolating cloned vehicle comprising said nucleic acid sequence of interest;

substituting the vehicle's origin of replication of a second kind of micro-organism by an origin of replication of said first kind of micro-organism; and

cloning the resulting vehicle in a culture of said first kind of micro-organism.

Preferably, said first kind of organism is different from said second kind of organism, e.g. they might require distinct factors to stably propagate a cloning vehicle. Most preferably, said first kind of organism belongs to another species and or genus as compared to said second kind of microorganism. A preferred embodiment therefore provides a method according to the invention, wherein said first kind of organism and said second kind of organism are different from each other. They preferably belong to different species and/or genera. With a method of the invention it has become possible to efficiently clone nucleic acid sequences of interest in micro-organisms that are less amenable to conventional nucleic acid manipulations, as compared to E.coli. The present invention enables high-throughput cloning (and, preferably, expressing) of a nucleic acid of interest in a first kind of micro-organism, while an initial (preferably high-throughput) cloning of the nucleic acid of interest is done in a second kind of micro-organism. Preferably, a second kind of organism is used for which an origin of replication (ori) is available in the art, and/or for which a selection marker, such as for instance an antibioticum selection marker, is available. Said second kind of micro-organism is preferably E.coli. Said first kind of micro-organism is preferably any micro-organism other then E.coli. The need of shuttle vectors is circumvented. With a method of the invention it has become possible to take advantage of the cloning properties of both said first and said second kind of micro-organism. Said second micro-organism is preferably a kind of micro-organism with a high cloning efficiency, such as E.coli. Said first kind of micro-organism preferably comprises an expression property of interest. Said first kind of micro-organism is for instance preferably capable of producing a protein of interest with a desired conformation.

In a first step of a method according to the invention a nucleic acid of interest is inserted into a nucleic acid vehicle, which vehicle comprises an origin of replication (ori) of a second kind of micro-organism. A nucleic acid vehicle is defined as a compound which comprises nucleic acid. Preferably, said nucleic acid vehicle consists of nucleic acid. The term “nucleic acid” encompasses sequences comprising natural nucleotides (adenine, guanine, cytosine, thymine and/or uracil) and/or non-natural nucleotides (such as for instance inosine). Artificial nucleic acid analogues, such as for instance—but not limited to—peptide nucleic acid (PNA), are also encompassed by the term “nucleic acid”.

Said nucleic acid vehicle preferably comprises a plasmid or a vector. In this first step, use is made of a nucleic acid vehicle which comprises an origin of replication of a second kind of micro-organism. Said origin of replication is a nucleic acid region which is necessary for initiation of replication of the nucleic acid vehicle. An origin of replication of a second kind of micro-organism is recognized by the nucleic acid replication machinery of said second kind of micro-organism. Different micro-organisms require different kinds of replication origins; an origin-recognition protein of a micro-organism (e.g. DnaA in E.coli) is capable of binding to an origin of replication. Upon recognition, replication is started, which require additional proteins that can be specific for a given micro-organism. Hence, an origin of replication has to be chosen which is recognized by a micro-organism of choice. If E.coli is chosen as a second kind of micro-organism, the E.coli origin of replication from pBR322, pSC101 or p15A is for instance used. If B. subtilis is chosen as a second kind of micro-organism, the B. subtilis origin of replication from pAMβ1 or pC194 is for instance used. However, any origin of replication of a micro-organism of choice is suitable.

A nucleic acid of interest is inserted into a nucleic acid vehicle using any cloning method available in the art. In a preferred embodiment, said nucleic acid of interest is inserted into said first vehicle and cloned in a culture of said second kind of micro-organism using a method selected from the group consisting of a Ligation Independent Cloning (LIC) procedure, Gateway, Univector Plasmid-fusion System (UPS), and methods that are derived from LIC such as for instance Enzyme-Free Cloning (EFC) or Sequence and Ligation Independent Cloning (SLIC). In one embodiment a ligation-independent cloning (LIC) procedure (Aslanidis and de Jong, 1990) is used. Contrary to methods that rely on recombination events (e.g., Gateway (Walhout et al, 2000) or the Univector Plasmid-fusion System (UPS) (Liu et al, 1998)), ligation-independent cloning is less restricted in the design of the sequences flanking the gene(s). Therefore, the cloning-related sequences attached to the recombinant protein(s) can be minimized. One embodiment of a LIC procedure is schematically outlined in FIG. 1B. This embodiment of a LIC cloning procedure involves linearization of a vector by restriction at a unique SwaI site in the middle of the LIC cassette, followed by T4 DNA polymerase treatment to create long, defined single-stranded overhangs, complementary to those of a nucleic acid sequence (for instance a PCR product). The resulting vector and nucleic acid sequence are mixed and transformed to an expression host, for instance by chemical transformation using CaCl₂. The LIC cassettes are preceded by promoter regions specific for the intended expression host. Ligation-independent cloning of nucleic acid products proved highly efficient for E. coli vectors, indicating that the length and composition of the complementary overhangs of vector and insert suffice for formation of stable heteroduplexes. In contrast, direct transformation of L. lactis with a stable heteroduplex of a LIC vector and a compatible insert yielded no or only very few positive clones. Therefore, a second micro-organism is preferably chosen which allows for efficient cloning, such as—but not limited to—E.coli.

In a second step of a method according to the invention, cloned vehicle comprising a nucleic acid of interest is isolated. This is done using any nucleic acid isolation method known in the art, such as for instance the method of Birnboim and Doly (1979).

Subsequently, the origin of replication of the cloned vehicle is substituted by an origin of replication of a first micro-organism. This is preferably done using a recombinase or, even more preferably, using a restriction enzyme. In that case the vehicle is designed such that a first recombinase site or restriction enzyme recognition sequence is present before the origin of replication of a second micro-organism. A second recombinase site or restriction enzyme recognition sequence is preferably present after said origin of replication. Upon incubation with a recombinase or a restriction enzyme recognizing said sequences, the origin of replication is cut out of the vehicle. Subsequently, an origin of replication of a first micro-organism is inserted into the vehicle. The resulting vehicle is preferably essentially devoid of elements derived from said second kind of micro-organism in order to allow efficient cloning and maximal stability of the vehicle in said first kind of micro-organism. Hence, with a method according to the invention, the use of shuttle vectors is not necessary. This is advantageous because any additional sequence increases the size of the resulting vector. In order to be capable of efficient cloning, the size of the resulting vehicle is preferably as small as possible. Moreover, smaller vehicles are more stable. Hence, the presence of additional, foreign sequences unnecessarily enlarges the resulting vehicle, decreases the stability and decreases the maximum size that a nucleic acid sequence of interest is allowed to have. An embodiment of the present invention wherein the resulting vehicle is essentially devoid of elements derived from said second kind of micro-organism is therefore preferred. By essentially devoid is meant that the resulting vehicle comprises no more than 15, preferably no more than 10 nucleotides derived from said second kind of micro-organism. Moreover, the resulting vehicle preferably comprises no more than 100, more preferably no more than 50, most preferably no more than 30 “foreign” nucleotides originating from the recombinase recognition sites and/or restriction enzyme recognition sites of the original vehicle(s).

A non-limiting, preferred embodiment is schematically shown in FIG. 1A. According to this embodiment, a nucleic acid of interest is inserted into a first vehicle comprising an E.coli origin of replication. Said vehicle is amplified in E.coli. Subsequently, the vector is isolated and cut by the SfiI restriction enzyme, preferably in the presence of another vector comprising an L.lactis origin of replication which is flanked by similar SfiI sites. Upon treatment with T4 DNA ligase and ATP, the E.coli origin of replication of the first vehicle is subsequently replaced by the L.lactis origin of replication in a significant part of the vehicles. Of course, many alternative embodiments are within the scope of the present invention. Any way of exchanging any origins of replication is suitable.

In a subsequent step of a method according to the invention, resulting nucleic acid vehicles comprising a nucleic acid sequence of interest and an origin of replication of a first micro-organism are transformed in said first kind of micro-organism. Said nucleic acid sequence of interest is preferably expressed in said first kind of micro-organism. This way, efficient cloning and expression of a nucleic acid of interest in any micro-organism for which plasmids are available has become feasible. Since a method of the invention allows the use of a second kind of micro-organism more amenable to (high-throughput) cloning, the cloning efficiency limitations of many first kinds of micro-organisms are at least in part bypassed.

In a preferred embodiment at least two nucleic acid vehicles are used. One vehicle comprises an origin of replication of a first kind of micro-organism and one vehicle comprises an origin of replication of a second kind of micro-organism. A nucleic acid sequence of interest is inserted into the vehicle comprising an origin of replication of a second kind of micro-organism and the resulting vehicle is transformed in said second kind of micro-organism. Subsequently, the origin of replication of the vehicle containing the nucleic acid sequence of interest is replaced by the origin of replication of the other vehicle and the resulting vehicle is subsequently transformed in the first kind of micro-organism. A preferred embodiment thus provides a method according to the invention comprising:

providing a first nucleic acid vehicle which comprises an origin of replication of said second kind of micro-organism;

introducing a nucleic acid sequence of interest into said first vehicle;

providing a second nucleic acid vehicle which comprises an origin of replication of said first kind of micro-organism; and

substituting a part of said first vehicle, which part comprises said origin of replication of said second micro-organism but not said nucleic acid of interest, by a part of said second vehicle, said part comprising said origin of replication of said first microorganism.

Preferably said second vehicle comprises a complete origin of replication of said first kind of micro-organism. In one alternative embodiment said second vehicle comprises a part of an origin of replication of said first kind of micro-organism. In this embodiment, said first vehicle preferably also comprises a part of said origin of replication of said first kind of micro-organism, which part is preferably not present in said second vehicle. After a nucleic acid of interest has been inserted into said first vehicle, a sequence of said second vehicle comprising said part of an origin of replication of said first micro-organism is preferably inserted into said first vehicle. In one embodiment a part of said origin of replication of said first micro-organism which is present in said first vehicle and a part of said origin of replication of said first micro-organism which is present in said second vehicle are functionally linked together, so that said parts are capable of acting together. Said second vehicle preferably comprises a significant part, more preferably a functional part, of said origin of replication of said first kind of micro-organism.

The invention also provides means and methods for even further optimizing nucleic acid manipulation efficiency. The present inventors have recognized that the use of restriction enzymes that produce different, non-palindromic overhangs that are not compatible with each other is preferred in order to enhance cloning efficiency. Non-limiting examples of such restriction enzymes are SfiI, SapI, Ksp632I, AarI, BglI, PflMI and BstXI. Said restriction enzymes preferably recognize nucleic acid sequences that rarely occur in the genomes of prokaryotes and eukaryotes, so that unintentional cutting of nucleic acid inserts of interest is best avoided. In principle, the more specific base pairs a recognition site has, the smaller the chance is that it occurs in a given nucleic acid sequence. The recognition sites of Ksp632I, BglI, PflMI and BstXI have six specific base pairs, the recognition sites of SapI and AarI have seven specific base pairs and the recognition site of SfiI has eight specific base pairs. Therefore, in order to even better avoid unintentional cutting of nucleic acid insert sequences of interest, a method according to the invention is preferably performed using SapI, AarI or SfiI. Most preferably, SfiI is used. The use of SfiI is particularly preferred because for other restriction enzymes available in the art that produce different, non-palindromic overhangs that are not compatible with each other, the occurrence of recognized nucleic acid sequences in genomes is more frequent and/or the maximal combinations of protruding sequences is less than that of SfiI. Therefore, the application of SfiI is particularly efficient. Furthermore, a restriction enzyme that is active in the same kind of buffer wherein T4 DNA ligase is active is preferred, so that in one embodiment cleavage reactions with at least one restriction enzyme and ligase reactions with T4 DNA ligase are carried out without need to change the reaction buffer, mere supplementation of the buffer with T4 ligase and ATP would suffice to start the ligation. SfiI is active in the same kind of buffer wherein T4 DNA ligase is active. Therefore, SfiI is also preferred because of this property.

By compatible is meant that two nucleic acid sequences are capable of binding each other because they comprise single stranded complementary sequences of a sufficient length. Hence, nucleic acid sequences that are not compatible with each other are not capable of annealing to each other because they do not comprise single stranded, complementary sequences of a sufficient length.

A particularly preferred restriction enzyme is SfiI. SfiI recognizes the sequence 5′ GGCCNNNNNGGCC 3′ (N being any nucleotide). The sequence is cut at:

5′ GGCCNNNN \hat{ } NGGCC 3′
3′ CCGGN \hat{ } NNNNCCGG 5′

As a result, an overhang of NNN is produced. Hence, as indicated in FIG. 1C, nucleic acid cleavage by SfiI generates a 3′ overhang that can be composed of any combination of three nucleotides. In a preferred embodiment of a method of the present invention, a first nucleic acid vehicle is used which comprises preferably two different SfiI sites, flanking an origin of replication of a second kind of micro-organism, which SfiI sites, upon incubation with SfiI, yield different, non-palindromic overhangs that are not compatible with each other. Incubation with SfiI results in a cut vehicle which is devoid of the origin of replication of said second micro-organism. Two ends of this vehicle that has been cut by SfiI do not anneal to each other. According to a further preferred embodiment, said two ends are capable of annealing to a second nucleic acid comprising a different origin of replication. This way, said second nucleic acid is very efficiently introduced into said vehicle.

Said second nucleic acid comprising a different origin of replication preferably originates from a second nucleic acid vehicle comprising said origin of replication, flanked by SfiI sites. The SfiI sites are chosen such that the resulting overhangs are compatible with the SfiI overhangs of the above mentioned vehicle that has been cut by SfiI. Upon incubation with SfiI, the second vehicle is cut and a nucleic acid sequence is obtained which comprises an origin of replication of a first micro-organism with SfiI overhangs capable of annealing to said first vehicle which has been cut by SfiI.

A preferred embodiment thus provides a method for cloning a nucleic acid sequence of interest in a first kind of micro-organism, the method comprising:

providing a first nucleic acid vehicle which comprises an origin of replication of said second kind of micro-organism;

introducing a nucleic acid sequence of interest into said first vehicle;

providing a second nucleic acid vehicle which comprises an origin of replication of said first kind of micro-organism; and

substituting a part of said first vehicle, which part comprises said origin of replication of said second micro-organism but not said nucleic acid of interest, by a part of said second vehicle, said part comprising said origin of replication of said first microorganism, wherein said part of said first vehicle has different, non-palindromic overhangs that are not compatible with each other. Preferably, said overhangs are compatible with the overhangs of said part of said second vehicle.

Said parts are preferably obtained by cleavage of said vehicles by SfiI.

An advantage of a method according to the invention is the fact that manipulations of said first and second vehicle are carried out without the need for purification of the vehicle fragments. Hence, methods according to the invention allow for minimal sample handling and automation of the methods.

A method of the invention involves transformation of nucleic acid sequences of interest to micro-organisms. In order to select micro-organisms that comprise the nucleic acid sequences of interest, selection markers are preferably used. Preferably, a nucleic acid sequence comprising an origin of replication of a first kind of micro-organism together with a first selection marker is used. Additionally, or alternatively, a nucleic acid sequence comprising an origin of replication of a second kind of micro-organism together with a second selection marker is preferably used. A non-limiting example of a suitable selection marker is a gene which provides resistance against the action of an enzyme and/or antibiotic, such as for instance a chloramphenicol resistance gene or a beta-lactam antibiotic resistance gene. Alternatively, or additionally, a selection marker is used that complements an auxotrophy (e.g. biosynthetic deficiency) in the microorganism in which the nucleic acid sequences are preferably expressed, such as for instance the alr gene, encoding an alanine racemase (Bron et al, 2002), or allows selection based on alternative selection procedures, such as for instance the shsp gene, encoding a small heat shock protein (El Demerdash et al, 2003). If a nucleic acid sequence is coupled to a selection marker, the presence of said nucleic acid sequence in a micro-organism is determined by determining whether the selection marker is present. For instance, an origin of replication of a second kind of micro-organism is coupled to a beta-lactamase resistance gene. After transformation, the resulting micro-organisms are propagated in the presence of beta-lactamase. Micro-organisms that are propagated apparently carry the beta-lactamase resistance gene which indicates that they also carry the origin of replication of a second kind of micro-organism.

A non-limiting example of the use of selection markers is schematically depicted in FIG. 1A. A vector comprising an E.coli origin of replication is provided with a nucleic acid sequence of interest and transformed in E.coli. This vector has both a chloramphenicol resistance gene (cat) and a beta-lactamase resistance gene (bla). E.coli micro-organisms carrying this vector are resistant to beta-lactamase. Subsequently, the E.coli origin of replication is replaced by an L.lactis origin of replication. The resulting vector does no longer comprise the beta-lactamase resistance gene, but does contain the L.lactis origin of replication. Hence, L.lactis clones that are resistant to chloramphenicol contain a nucleic acid vehicle containing both the chloramphenicol resistance gene and the L.lactis origin of replication which allows propagation of the nucleic acid in L.lactis. Of course, many other (combinations of) selection markers are suitable for selecting micro-organisms of interest.

Further provided is therefore a method for cloning a nucleic acid sequence of interest in a first kind of micro-organism, the method comprising:

providing a first nucleic acid vehicle which comprises an origin of replication of a second kind of micro-organism;

introducing a nucleic acid sequence of interest into said first vehicle;

providing a second nucleic acid vehicle which comprises an origin of replication of said first kind of micro-organism; and

substituting a part of said first vehicle, which part comprises said origin of replication of said second micro-organism but not said nucleic acid of interest, by a part of said second vehicle, said part comprising said origin of replication of said first microorganism, wherein said part of the first vehicle comprising an origin of replication of said second micro-organism comprises a selection marker, such as for instance a resistance gene. In one embodiment said second vehicle comprising an origin of replication of said first micro-organism comprises a different selection marker, such as for instance a different resistance gene.

A method according to the present invention is particularly suitable for improving the efficiency of cloning and expressing a nucleic acid sequence of interest in a micro-organism of interest. Preferably, a first cloning step is performed wherein a nucleic acid of interest is efficiently inserted into a nucleic acid vehicle and cloned in another kind of micro-organism which has better cloning properties as compared to the micro-organism wherein the nucleic acid of interest is finally expressed. Preferably, a second kind of organism is used which is capable of more efficiently cloning a nucleic acid of interest as compared to said first kind of micro-organism. For instance, a second kind of organism for which an origin of replication (ori) is available in the art, and/or for which a selection marker, such as for instance an antibioticum selection marker, is available in the art, is preferably used. One embodiment thus provides a method for cloning a nucleic acid sequence of interest in a first kind of micro-organism, the method comprising:

providing a first nucleic acid vehicle which comprises an origin of replication of a second kind of micro-organism;

introducing a nucleic acid sequence of interest into said first vehicle;

cloning said vehicle in a culture of said second kind of micro-organism;

providing a second nucleic acid vehicle which comprises an origin of replication of said first kind of micro-organism;

substituting a part of said first vehicle, which part comprises said origin of replication of said second micro-organism but not said nucleic acid of interest, by a part of said second vehicle, said part comprising said origin of replication of said first microorganism; and

cloning the resulting vehicle in a culture of said first kind of micro-organism;

wherein the process of inserting said nucleic acid of interest into said first vehicle and cloning it in a culture of said second kind of micro-organism is more efficient as compared to a process of inserting said nucleic acid of interest into said second vehicle and cloning it in a culture of said first kind of micro-organism.

In one preferred embodiment said first and said second kind of micro-organisms are bacteria. Preferably, said second kind of micro-organism is E.coli and said first kind of micro-organism is a micro-organism different from E.coli.

A method according to the invention is particularly suitable for efficient cloning of a nucleic acid of interest and expression of said nucleic acid of interest in a micro-organism for which efficient cloning methods are not available in the art. Such micro-organisms are called herein recalcitrant micro-organisms. Recalcitrant micro-organisms are defined herein as micro-organisms in which, with the technology currently available, it is difficult to clone a nucleic acid of interest. As used herein, it is typically difficult to clone a nucleic acid of interest if less than 1000 colony forming units per μg nucleic acid are formed during the cloning procedure. With recalcitrant micro-organisms it is difficult to make gene constructs at a medium- to high-throughput rate, meaning that transformation of these organisms with ligation mixtures generally results in low transformation efficiencies (generally less than 1000 colony forming units per μg DNA). As used herein, gene constructs encompass, but are not limited to, homologous genes and/or nucleic acid sequences to be inserted in a vehicle. In some embodiments, said gene constructs comprise nucleic acid sequences that have been mutated, for instance by error-prone PCR, and for which screening of large plasmid libraries is desired.

Contrary to prior art methods such as the methods disclosed in WO 96/40724 and WO 00/29000, which exchange nucleic acid sequences between vectors where after the resulting vector of interest is cloned in the commonly used E.coli bacteria, preferred methods according to the present invention comprise efficient cloning of a nucleic acid of interest and expression of said nucleic acid of interest in a micro-organism other then E.coli, preferably in recalcitrant bacteria, for which efficient cloning methods are not available in the art. With a method according to the invention high throughput expression procedures in recalcitrant micro-organisms have become possible.

In Table 3 a non-limiting list of well known recalcitrant micro-organisms available in the art is given. However, many more recalcitrant micro-organisms are known. Nucleic acid manipulation and/or nucleic acid cloning in recalcitrant micro-organisms is more difficult and/or less efficient as compared to E.coli. For example, in a recent study the cloning of genes in E. coli and L. lactis were compared, using existing technology (Surade et al, 2006). For E. coli the success rate was 216 out of 222, whereas for L. lactis the success rate was only 39 out of 71. Thus, even after numerous attempts the authors of Surade et al failed to clone 32 genes in L.lactis.

Contrary, much better results are obtained if a method according to the present invention is applied. For instance, as shown in the Examples, in embodiments of the invention wherein said first kind of micro-organism is L.lactis and wherein said second kind of micro-organism is E.coli, the initial cloning step has the same, high efficiency as standard E. coli vectors; for the performed procedures according to the invention thus far not a single failure in the conversion process has been observed, with a current count of over 300 gene constructs.

A preferred embodiment of the present invention therefore provides a method according to the invention wherein said first kind of micro-organism is a recalcitrant micro-organism. In one embodiment said first kind of micro-organism is one of the well known micro-organisms listed in Table 3.

In one embodiment said first micro-organism is for instance Lactococcus lactis, Streptomyces sp or Sulfolobus solfataricus but most microorganisms known to date are relevant in this context. Micro-organisms other then E.coli are often preferred because various proteins are better expressed by these micro-organisms as compared to E.coli. For instance, various proteins obtain a better conformation and/or post-translational modification(s) as a result of the cellular environment of other micro-organisms, as compared to E.coli. Moreover, additional parameters such as their codon usage, types and quantities of chaperones, resistance to expression of proteins which are toxic to E. coli, and lipid composition of the plasma membrane is often more favorable to protein expression in a functional state as compared to E.coli. Micro-organisms other than E.coli are also preferred for expressing proteins if the produced proteins are more stable in their close to own cellular environment. Hence, even though a conventional cloning procedure such as a LIC procedure is much less efficient in for instance L.Lactis, Streptomyces sp or S. solfataricus, as compared to E.coli, it is often still preferable to clone and express a nucleic acid sequence in micro-orgenisms other then E.coli. With a method according to the invention, efficient cloning and expression of nucleic acid sequences in recalcitrant micro-organisms has become possible.

A method according to the invention is preferably provided wherein said second micro-organism is Escherichia coli, because this micro-organism is known to have high transformation efficiencies and various gene manipulation tools have been specifically developed and optimized for this organism. This is amongst other things due to the fact that suitable origins of replications and plasmid systems exist in the art. Insertion of nucleic acids of interest into such plasmid systems and cloning of the resulting plasmids have proven to be very efficient in E.coli. This micro-organism is therefore very suitable for use in a first cloning step of a method according to the invention.

Hence, in a particularly preferred embodiment, second micro-organism is Escherichia coli. As demonstrated in the Examples, E.coli is very suitable for efficient nucleic acid cloning during the first phase of a method according to the invention.

A method according to the invention is particularly suitable for efficient cloning and expressing a nucleic acid sequence of interest by a micro-organism of choice, preferably by a recalcitrant micro-organism. A method according to the invention, further comprising allowing expression of said nucleic acid of interest by a culture of said first kind of micro-organism is therefore also herewith provided. Once a nucleic acid of interest is expressed in a micro-organism with a method according to the invention, such expression product is preferably obtained for further use. For instance, a therapeutic protein is produced, isolated and used for the preparation of a medicament. Many methods for obtaining, isolating and/or purifying protein that has been produced by micro-organisms are known in the art. Non-limiting examples include the use of affinity tags such as oligo-His-tags in combination with metal-chelate affinity chromatography, MBP-tags in combination with amylose-resin affinity chromatography and other generic chromatography methods to purify proteins. A method according to the invention, further comprising obtaining an expression product of said nucleic acid sequence of interest is therefore also herewith provided, as well as an expression product obtained by a method according to the invention.

In one embodiment said nucleic acid of interest encodes a multidomain protein and/or a membrane protein. Heterologous expression of such proteins in a functionally competent state is often problematic in the well-established expression hosts E.coli and yeast. With a method of the invention, however, efficient cloning and expression of nucleic acid sequences encoding such proteins in alternative expression hosts has become possible.

Now that it has become possible to efficiently clone and express nucleic acid sequences in a wide variety of micro-organisms, a method of the invention is performed for many applications. For instance, nucleic acid sequences are mutated (for instance using error-prone PCR) and the resulting nucleic acid sequences are cloned and expressed. High-throughput screening of mutated proteins in order to look for improved variants has become possible. Micro-organisms other than the well-established E.coli are now used for high-throughput expression. Mutant proteins that do not obtain a desired conformation or post-translational modification in E.coli are now efficiently expressed using other kinds of micro-organisms. A cloning assay wherein a nucleic acid of interest is cloned and expressed by a method according to the invention is therefore also provided. Said cloning assay is preferably a high-throughput cloning assay.

Further provided is a kit of parts comprising:

a first nucleic acid vehicle which comprises an origin of replication of a second kind of micro-organism; and

a second nucleic acid vehicle which comprises an origin of replication of a first kind of micro-organism;

wherein each vehicle comprises at least two recombinase sites and/or restriction enzyme cleavage sites wherein said recombinase sites and/or restriction enzyme cleavage sites are preferably not present within said origins of replication. This way, the origins of replication are cut out of the vector and exchanged by the other kind of origin of replication at will.

Preferably, each vehicle comprises at least two restriction enzyme cleavage sites which, upon cleaving, yield two different, non-palindromic overhangs that are not compatible with each other so that two ends of a vehicle that has been cut do not anneal to each other. This way, cloning efficiency is even further enhanced. When non-complementary, non-palindromic overhangs are used, a nucleic acid sequence of interest is in principle only capable of being inserted into a nucleic acid vehicle in one direction. Non-complementary, non-palindromic overhangs are therefore preferred in order to obtain a nucleic acid vehicle with a nucleic acid sequence of interest in the right orientation. Preferably, said first kind of micro-organism is different from said second kind of micro-organism. More preferably, said first kind of micro-organism belongs to another species and or genus as compared to said second kind of micro-organism. Said first kind of micro-organism preferably comprises a micro-organism other then E.coli. In a particularly preferred embodiment said first kind of micro-organism is a recalcitrant micro-organism.

In one embodiment said first nucleic acid vehicle comprises a sequence encoding an affinity tag (such as for instance a His-tag) and/or another kind of fusion partner (such as for instance GFP), which affinity tag or fusion partner will become attached to the protein(s) encoded by the nucleic acid sequence of interest. Such affinity tag or fusion partner facilitates isolation, and/or detection, and/or purification of said protein(s).

Such kit of parts is particularly suitable for performing a method according to the invention, wherein a nucleic acid sequence of interest is introduced into said first vehicle, where after the resulting vehicle is cloned in a culture of said second kind of micro-organism, where after the cloned vehicle's origin of replication is replaced by the origin of replication of said second vehicle, where after the resulting vehicle is cloned in a culture of said first kind of micro-organism. Said origins of replications are preferably separated from the vehicles where they are originally present by the action of a recombinase or one or more restriction enzymes. Therefore, recombinase sites and/or restriction enzyme cleavage sites are preferably present in the flanking regions of said origins of replication.

A kit of parts according to the invention preferably comprises a first and a second vehicle, wherein each vehicle comprises the same kind of restriction enzyme cleavage sites which, upon cleaving, yield two different, non-palindromic overhangs that are not compatible with each other so that two ends of a vehicle that has been cut do not anneal to each other. This way, cloning efficiency is even further enhanced. The restriction enzyme sites of the vehicles are preferably chosen such that, upon incubation with said restriction enzyme, the parts of the vehicles that are intended to be ligated to each other have complementary single stranded overhangs. Preferably, at least one vehicle of a kit of parts according to the invention has at least two SfiI cleavage sites. Most preferably, all vehicles of a kit of parts according to the invention have at least two SfiI cleavage sites.

One preferred embodiment provides a kit of parts according to the invention, wherein said first vehicle comprises an Escherichia coli origin of replication. As already outlined before, E.coli is particularly suitable for the first cloning step of a method according to the invention. In one embodiment said Escherichia coli origin of replication is derived from pBR322.

The second vehicle of a kit of parts according to the invention preferably comprises an origin of replication of a micro-organism other then E.coli, preferably an origin of replication of a recalcitrant micro-organism. These micro-organisms are preferred for the production of various kinds of (human) proteins, as discussed before. In one embodiment a Lactococcus lactis origin of replication is used which is derived from pSH71.

The first vehicle of a kit of parts according to the invention preferably comprises a selection marker, such as for instance an antibiotic resistance gene. Furthermore, the second vehicle of said kit of parts preferably comprises a selection marker, such as for instance an antibiotic resistance gene. Selection markers are used for selection of micro-organisms which carry the nucleic acid vehicle, as explained herein before.

With a method according to the invention it has become possible to efficiently clone a nucleic acid of interest using a kind of micro-organism which is less amenable to conventional nucleic acid manipulations. For instance, a Lactococcus lactis culture has been obtained wherein the amount of colony forming units per microgram nucleic acid which was used in said method is at least 10⁵ CFU/g DNA.

Further provided is therefore a Lactococcus lactis culture obtainable by a method according to the invention, wherein the amount of colony forming units per microgram nucleic acid of interest which was used in said method is at least 10⁵ CFU/g DNA.

The invention also provides a recombinant micro-organism comprising a nucleic acid vehicle, which vehicle comprises:

an origin of replication of said kind of micro-organism;

a nucleic acid sequence of interest; and

at least two restriction enzyme cleavage sites which, upon cleaving, yield two different, non-palindromic overhangs that are not compatible with each other and wherein said restriction enzyme cleavage sites are not present within said origins of replication. Said micro-organism is preferably a micro-organism other then E.coli. In one preferred embodiment said micro-organism is a recalcitrant micro-organism. In one embodiment said micro-organism is Lactococcus lactis.

Moreover, said restriction enzyme cleavage sites are preferably SfiI, SapI, Ksp632I, AarI, BglI, PflMI and/or BstXI cleavage sites, more preferably SapI, AarI or SfiI cleavage sites. Most preferably, said restriction enzyme cleavage sites are SfiI cleavage sites. As described herein before, the use of SfiI, SapI, Ksp632I, AarI, BglI, PflMI and/or BstXI allows cleavage of a nucleic acid vehicle, whereby two ends of a vehicle that has been cut do not anneal to each other. SfiI, SapI, Ksp632I, AarI, BglI, PflMI and/or BstXI cleavage sites are designed such that the parts of two different vehicles that are intended to be ligated to each other have complementary single stranded overhangs. The use of SfiI further provides the additional advantage that it is active in the same kind of buffer wherein T4 DNA ligase is active. Hence, in a preferred embodiment wherein T4 DNA ligase and SfiI are used, the cleavage reactions and ligase reactions are carried out without need to change the reaction buffer (meaning that actions other than the administration of SfiI, T4 DNA ligase and ATP are not necessary).

The invention is further explained in the following examples. These examples do not limit the scope of the invention, but merely serve to clarify the invention.

### Examples

We now present a generic cloning strategy, compatible with high-throughput manipulations, that generates a native plasmid vector optimal for the expression host and devoid of alien (e.g., E. coli-derived) elements. The Vector Backbone Exchange (VBEx) procedure presented here is specific for cloning in L. lactis but readily adaptable for all organisms for which a plasmid, selection marker and transformation method are available. VBEx has been used to generate over 300 gene constructs, and gene cloning up to expression screening in L. lactis has been carried out at a rate of 48 per week.

To facilitate the initial steps in the cloning of large numbers of open reading frames, we employed a ligation-independent cloning (LIC) procedure (Aslanidis and de Jong, 1990). Contrary to methods that rely on recombination events (e.g., Gateway (Walhout et al, 2000) or the Univector Plasmid-fusion System (UPS) (Liu et al, 1998)), ligation-independent cloning is less restricted in the design of the sequences flanking the gene(s). Therefore, the cloning-related sequences attached to the recombinant protein(s) can be minimized. The cloning procedure involves linearization of the vector by restriction at a unique SwaI site in the middle of the LIC cassette, followed by T4 DNA polymerase treatment to create long, defined single-stranded overhangs, complementary to those of the PCR product (FIG. 1B). The LIC cassettes are preceded by promoter regions specific for the intended expression host. Here, we used the P_{BAD} and P_{NIS} promoters of the E. coli and L. lactis expression plasmids pBAD24 (Guzman et al, 1995) and pNZ8048 (de Ruyter et al, 1996), respectively. Ligation-independent cloning of PCR products proved highly efficient for the E. coli vectors, indicating that the length and composition of the complementary overhangs of vector and insert suffice for formation of stable heteroduplexes. In contrast, direct transformation of L. lactis with the stable heteroduplex of the pNZ8048-derived LIC vector and a compatible insert yielded no or only very few positive clones (data not shown).

To overcome the poor cloning efficiencies in L. lactis, a new strategy (VBEx) was devised that allowed the initial (high-throughput) cloning to be done in E. coli, but avoided the use of shuttle vectors. Although we have used VBEx in combination with LIC, the strategy is fully compatible with Gateway, UPS and variations on LIC (e.g, Enzyme Free Cloning (de Jong et al, 2007) and SLIC (Li and Elledge, 2007)). The method relies on the bisection of a bona fide plasmid vector of the expression host into two parts, thereby separating the selection marker and the origin of replication. For L. lactis, the origin of replication of the pNZ8048 plasmid (pSH71 replicon) was separated from the chloramphenicol resistance gene (cat) (FIG. 1a). The segment containing the cat and LIC sequence was fused to the backbone of a vector containing an E. coli origin of replication and the β-lactamase resistance gene (bla). The resulting plasmid, pRExLIC, allows the LIC manipulation to take place in E. coli. The segment containing the L. lactis origin of replication was fused to an erythromycin selection marker (yielding plasmid pERL), which enables replication and selection in the expression host.

Rapid and high-throughput compatible reconstruction of the original L.lactis expression plasmid from the relevant segments of pRExLIC and pERL is assured by flanking the ends of each half with distinct SfiI restriction sites (SfiIx and SfiIy in FIG. 1a). DNA cleavage by SfiI generates a 3′ overhang that can be composed of any combination of three nucleotides. The two SfiI sites used, yield different, non-palindromic overhangs that are not compatible with each other (FIG. 1c). The combination of i) both halves of the original expression vector having properties that can be selected for, and ii) different 3′ overhangs after SfiI digestion at either side of each plasmid segment, ensures minimal sample handling and allows automation of the method. As each half of the expression vector has unique selectable properties, no gel electrophoresis and purification of the SfiI-fragments is required. Selection for the ability of the plasmid to replicate in L. lactis in the presence of chloramphenicol permits unique recovery of only the original expression vector from a complex mixture of SfiI-digested pRExLIC and pERL plasmids. Furthermore, no change of buffers between the digestion and ligation reaction is needed. Mere supplementation of the SfiI-buffer with ATP suffices for the T4 DNA ligase to be fully active.

In practice, the whole procedure can take place in a single tube in 3 hours. After joined SfiI digestion of pRExLIC and pERL (80 min at 50° C.), and subsequent heat inactivation of the restriction enzyme (20 min at 80° C.), ligation (60 min at 20° C.) of the fragments is started by the addition of ATP and T4 DNA ligase. Upon thermal inactivation of the T4 DNA ligase (20 min at 65° C.), aliquots of the mixture can be used for (electro-)transformation without further purification (FIG. 1). For L. lactis, we routinely observe high, reproducible transformation efficiencies (˜10⁶ CFU/μg DNA), in comparison to the low, variable efficiencies of traditional restriction-ligation cloning (˜10² CFU/μg DNA; e.g. see Surade et al, 2006)). Notably, with the VBEx procedure, all L. lactis transformants obtained carry the gene of interest inserted in pNZxLIC. Using this system, we generated over 300 expression constructs and assessed protein levels at a rate of 48 constructs per week without robotics. The overexpression of a small subset of membrane proteins in L. lactis is presented in FIG. 1d.

Importantly, DNA sequences from virtually all sequenced genomes are compatible with the cloning strategy, because SfiI sites (5′ GGCCNNNN̂NGGCC 3′) are rare (FIG. 2a). Analysis of all predicted gene transcripts of 492 archaeal and bacterial chromosomes and 30 eukaryotic genomes, present in the NCBI databank (February 2007) and Ensembl (release 43), respectively, indicated that well over 92% of these transcripts do not contain any SfiI site (Table 1). Moreover, use of the method is not limited to transcripts free of SfiI sites. As 64 different 3′ overhangs may be generated after SfiI digestion, internal SfiI sites with 3′ overhangs not matching those of the vector will result in a three or more-way ligation, but not form a bottleneck for the procedure. If needed, the vector can be readily adapted to use non-occuring or extremely rare overhangs. In 89% of the genomes analyzed at least two types of SfiI overhangs do not occur (FIG. 2b). The remaining 11% of the genomes contain several types of low occurrence SfiI overhangs.

To ensure the generality of the presented strategy towards inserts of different size, we compared SfiI digestion rates of pRExLIC derivatives holding inserts up to 3.7 kb (data not shown) and observed complete digestion after 80 min incubation. Furthermore, we demonstrated the absence of expression from the P_{NIS} promoter in the cloning host E. coli, using a sensitive activity based assay (data not shown). The success of assembly and stability in the pRExLIC and pNZxLIC vectors of recombinant DNA from bacterial, plant and mammalian origin proved very high and gene rearrangements have so far not been observed (n>300).

In summary, we have developed LIC-VBEx, a high-throughput compatible cloning system for L. lactis with unprecedented high efficiency, which can be readily adapted for any other expression host. Problems arising from shuttle vectors are avoided by using genuine expression plasmids. The LIC cassettes developed allow the tagging of the protein of interest with a cleavable decaHis-tag at either the N- or C-terminus (vectors holding these cassettes and derivatives with alternative tags or fusion-partners are shown in Materials and Methods section). The procedure described has thus far been used in our lab to prepare over 300 gene constructs with high efficiencies (˜90% for LIC; 100% for VBEx (data not shown)). In a non-automated setting, the full procedure from cloning to expression screening in E. coli and L. lactis, took place at a rate of approximately 48 constructs per week.

### Materials and Methods

Ligation-Independent Cloning (LIC). Inserts were amplified using Phusion DNA polymerase (Finnzymes) and gene specific primers extended at the 5′ side with LIC specific tails (Table A). Vectors containing a LIC cassette were purified using a plasmid isolation kit (Wizard® Plus, Promega). DNA was additionally purified by extraction with phenol:chloroform and chloroform to remove tracer amounts of proteins. Vectors (˜5 μg of DNA) were digested overnight with 25 units of SwaI (Roche) at 25° C. PCR products and digested vectors were subsequently gel purified (GFX PCR DNA & Gel Band Purification Kit, GE), eluted in 10 mM Tris-HCl, pH 7.5, 0.2 mM Na-EDTA, and stored at 4° C.

200 ng of SwaI-digested vector and equimolar quantities of inserts were treated separately with 0.5 U T4 DNA polymerase (Roche) at 20° C. for 30 min in the presence of 2.5 mM dCTP and 2.5 mM dGTP, respectively, followed by heat inactivation of the T4 DNA polymerase (20 min at 75° C.). The material, now in a “LIC-ready” state, can be stored at 4° C. for prolonged periods (over 6 months). LIC-ready vector (1 μl) and insert (3 μl) were mixed and after a 5 min incubation at RT transformed to 75 μl chemically-competent E. coli MC1061. Cells were plated on Luria Broth supplemented with ampicillin (100 μg/ml).

TABLE A
Primer extensions required for Ligation
Independent Cloning.

| Type of primer | Sequence (5′→3′) |
|---|---|
| nLIC forward | AT GGT GAG AAT TTA TAT TTT |
| | CAA GGT + gene specific |
| nLIC reverse | T GGG AGG GTG GGA TTT TCA TTA + |
| | gene specific |
| cLIC forward | ATG GGT GGT GGA TTT GCT + gene |
| | specific |
| cLIC reverse | TTG GAA GTA TAA ATT TTC + gene |
| | specific |

Vector Backbone Exchange. Exchange of the vector backbone of pRExLIC-derived vectors was done in a small volume (10 μl) in a PCR machine with heated lid to avoid condensation. The pERL vector (containing the L. lactis origin of replication) and a pRExLIC-derived vector (containing the L. lactis cat gene) were mixed (˜125 ng each) and the volume was adjusted to 10 μl by adding 1 μl 10× buffer (100 mM Tris-HCl, pH 7.5, 100 mM MgCl₂, 500 mM NaCl, 1 mg/ml BSA), 5 U SfiI (Fermentas) and sufficient milliQ. The sample was incubated for 80 min at 50° C., and 20 min at 80° C. to inactivate SfiI. After cooling to RT, ligation was started by the addition of 1.5 μl 8 mM Naz-ATP, pH 7, and 0.5 U T4 DNA ligase (Roche). The sample was incubated for 1 hr at 20° C. and 20 min at 65° C. to heat inactivate the T4 DNA ligase. Subsequently, 2 μl of the sample was transformed to 30 μl electrocompetent L. lactis NZ9000 (see below) and aliquots were plated on M17 plates (Terzaghi and Sandine, 1975) (Difco) supplemented with 0.5% glucose, 0.5 M sucrose, 5 μg/ml chloramphenicol. Parafilm-sealed plates were incubated at 30° C. until colonies appeared (˜18 hrs).

Electrotransformation of L.lactis. Preparation of electrocompetent L. lactis NZ9000 was essentially done as described (Holo and Nes, 1989; Wells et al, 1993), but with some critical modifications. Briefly, cells were grown in M17 supplemented with 0.5% glucose, 0.5 M sucrose and 2% glycine at 30° C. to OD₆₀₀=˜0.5. Cells were harvested by centrifugation at 5000×g for 15 min at 4° C. Following washes with 1 volume ice-cold solution A (0.5 M sucrose and 10% glycerol, prepared in milliQ), 0.5 volume solution A supplemented with 50 mM Na-EDTA, pH 7.5, and 0.25 volume solution A, cells were resuspended in 0.01 volume solution A. Aliquots of 40 μl were flash-frozen in liquid nitrogen and stored at −80° C. until use. For electroporation, cells were thawed on ice, combined with plasmid DNA, and transferred to an ice-cooled electroporation cuvet (2 mm gap). Cells were exposed to a single electrical pulse with a field strength of 2 kV, 25 μF capacitance and 200Ω resistance. Immediately following discharge, cells were mixed with 1 ml ice-cold M17 supplemented with 0.5% glucose, 0.5 M sucrose, 20 mM MgCl₂ and 2 mM CaCl₂, and left on ice for 10 min. Subsequently, cells were incubated at 30° C. for 2 hrs and aliquots were plated on M17 agar supplemented with 0.5% glucose, 0.5 M sucrose and 5 μg/ml chloramphenicol. was added. Plates were sealed and incubated overnight at 30° C.

TABLE 1
Analysis of the distribution of SfiI sites over pro- and
eukaryotic gene transcripts. For the pro- and eukaryotic
datapoints, 1.484.533 and 745.558 gene transcripts
were analyzed, respectively.

Percentage of transcripts

| SfiI sites in transcript | prokaryotes | eukaryotes |
|---|---|---|
| 0 | 92.18 | 92.06 |
| 1 | 6.42 | 6.49 |
| 2 | 1.09 | 1.04 |
| 3 | 0.22 | 0.23 |
| 4 | 0.06 | 0.08 |
| 5 | 0.02 | 0.03 |
| 6 | 0.01 | 0.02 |
| 7 | 0.00 | 0.01 |
| 8 | 0.00 | 0.01 |
| 9 | 0.00 | 0.01 |
| >9 | 0.00 | 0.02 |

Detailed Overview of the LIC Process.

nLIC cassette: A construct is made that contains an N-terminal 10 His-tag, followed by a TEV protease cleavage site and Your Favorite Protein (YFP). Using the 3′→5′ exonuclease activity of T4 DNA polymerase and dedicated tail-sequences, long defined overhangs are generated. These overhangs have a sufficiently high annealing temperature that mere mixing of complementary overhangs suffices in generating stable DNA sequences, ready for cell transformation.

The vector holds the nLIC cassette which contains a SwaI site. After SwaI digestion, the vector is treated with T4 DNA polymerase in the presence of dCTP, in order to generate the nLIC-ready overhangs (illustrated below).

1. nLIC cassette
N Met His His His His His His His His His His Gly Glu Asn Leu Tyr
5′ ATG CAT CAT CAC CAT CAT CAC CAT CAC CAT CAT GGT GAG AAT TTA TAT TTAAATCCCACCCTCCCAG
3′ TAC GTA GTA GTG GTA GTA GTG GTA GTG GTA GTA CCA CTC TTA AAT ATA AATTTAGGGTGGGAGGGTC
2. After digestion with SwaI
N Met His His His His His His His His His His Gly Glu Asn Leu Tyr
5′ ATG CAT CAT CAC CAT CAT CAC CAT CAC CAT CAT GGT GAG AAT TTA TAT TT AAATCCCACCCTCCCAG
3′ TAC GTA GTA GTG GTA GTA GTG GTA GTG GTA GTA CCA CTC TTA AAT ATA AA TTTAGGGTGGGAGGGTC
3. After treatment with T4 DNA polymerase + dCTP
N Met His His His His His His His His His His Gly Glu Asn Leu Tyr
5′ ATG CAT CAT CAC CAT CAT CAC CAT CAC CAT C AAATCCCACCCTCCCAG
3′ TAC GTA GTA GTG GTA GTA GTG GTA GTG GTA GTA CCA CTC TTA AAT ATA AA C

The insert is PCRed using primers with dedicated nLIC tails. After removal of primers and nucleotides, the insert is treated with T4 DNA polymerase in the presence of dGTP, in order to generate the nLIC-ready overhangs (illustrated below).

1. PCR
N is Gly Glu Asn Leu Tyr Phe Gln Gly Met X X
5′ AT GGT GAG AAT TTA TAT TTT CAA GGT ATG YFP TAA TGA AAA TCC CAC CCT CCC A
3′ TA CCA CTC TTA AAT ATA AAA GTT CCA TAC YFP ATT ACT TTT AGG GTG GGA GGG T
2. After treatment with T4 DNA polymerase + dGTP
N is Gly Glu Asn Leu Tyr Phe Gln Gly X X
5′ AT GGT GAG AAT TTA TAT TTT CAA GGT YFP TAA TG
3′ GTT CCA YFP ATT ACT TTT AGG GTG GGA GGG T

Subsequently, the nLIC-ready vector and insert are mixed. The defined overhangs anneal to stable structures with a Tₘ of approximately 44° C. and 58° C. for the 5′ and 3′ end of the gene, respectively. Small, 1 basepair gaps remain, which will be filled in vivo.

a. 5′ end, before annealing
N Met His His His His His His His His His H
5′ ATG CAT CAT CAC CAT CAT CAC CAT CAC CAT C
3′ TAC GTA GTA GTG GTA GTA GTG GTA GTG GTA GTA CCA CTC TTA AAT ATA AA.
is Gly Glu Asn Leu Tyr Phe Gln Gly
AT GGT GAG AAT TTA TAT TTT CAA GGT YFP
GTT CCA YFP
b. 5′ end, after annealing
N Met His His His His His His His His His His Gly Glu Asn Leu Tyr Phe Gln Gly
5′ ATG CAT CAT CAC CAT CAT CAC CAT CAC CAT CAT GGT GAG AAT TTA TAT TTT CAA GGT YFP
3′ TAC GTA GTA GTG GTA GTA GTG GTA GTG GTA GTA CCA CTC TTA AAT ATA AA. GGT CCA YFP
1. 3′ end, before annealing
N Met X X
5′ ATG YFP TAA TG AAA TCC CAC CCT CCC AG
3′ TAC YFP ATT ACT TTT AGG GTG GGA GGG T C
2. 3′ end, after annealing
N Met X X
5′ ATG YFP TAA TG. AAA TCC CAC CCT CCC AG
3′ TAC YFP ATT ACT TTT AGG GTG GGA GGG TC

cLIC cassette: A construct is made that contains a relatively small N-terminal modification (MGGGFA), Your Favorite Protein (YFP), and a C-terminal TEV protease cleavage site followed by a 10 His-tag. Using the 3′→5′ exonuclease activity of T4 DNA polymerase and dedicated tail-sequences, long defined overhangs are generated. These overhangs have a sufficiently high annealing temperature that mere mixing of complementary overhangs suffices in generating stable DNA sequences,ready for cell transformation. The vector holds the cLIC cassette which contains a SwaI site. After SwaI digestion, the vector is treated with T4 DNA polymerase in the presence of dCTP, in order to generate the nLIC-ready overhangs (illustrated below).

```
1. cLIC cassette
            M G G G F N L Y F Q G H H H H H H H H H H X
5′ C ATG GGT GGT GGA TTT A AAT TTA TAC TTC CAA GGT CAT CAT CAC CAT CAT CAC CAT CAC CAT CAT TAA
3′ G TAC CCA CCA CCT AAA T TTA AAT ATG AAG GTT CCA GTA GTA GTG GTA GTA GTG GTA GTG GTA GTA ATT
2. After digestion with SwaI
              M G G G F N L Y F Q G H H H H H H H H H H X
5′ C ATG GGT GGT GGA TTT A AAT TTA TAC TTC CAA GGT CAT CAT CAC CAT CAT CAC CAT CAC CAT CAT TAA
3′ G TAC CCA CCA CCT AAA T TTA AAT ATG AAG GTT CCA GTA GTA GTG GTA GTA GTG GTA GTG GTA GTA ATT
3. After treatment with T4 DNA polymerase + dCTP
            M G G G F N L Y F Q G H H H H H H H H H H X
5′ C A AAT TTA TAC TTC CAA GGT CAT CAT CAC CAT CAT CAC CAT CAC CAT CAT TAA
3′ G TAC CCA CCA CCT AAA CCA GTA GTA GTG GTA GTA GTG GTA GTG GTA GTA ATT
```

The insert is PCRed using primers with dedicated cLIC tails. After removal of primers and nucleotides, the insert is treated with T4 DNA polymerase in the presence of cGTP, in order to generate the cLIC-ready overhangs (illustrated below).

```
1. PCR
        M G G G F A E N L Y
5′ ATG GGT GGT GGA TTT GCT YFP GAA AAT TTA TAC
3′ TAC CCA CCA CCT AAA CGA YFP CTT TTA AAT ATG
  F Q
TTC CAA
AAG GTT
2. After treatment with T4 DNA polymerase + dGTP
        M G G G F A E N L Y
5′ ATG GGT GGT GGA TTT GCT YFP G
3′  GA YFP CTT TTA AAT ATG
  F Q
AAG GTT
```

Subsequently, the cLIC-ready vector and insert are mixed. The defined overhangs anneal to stable structures with a Tₘ of approximately 41° C. and 31° C. for the 5′ and 3′ end of the gene, respectively. Small, 1 basepair gaps remain, which will be filled in vivo.

```
a. 5′ end, before annealing
            M G G G F A
5′ C ATG GGT GGT GGA TTT GCT YFP
3′ G TAC CCA CCA CCT AAA GA YFP
b. 5′ end, after annealing
            M G G G F A
5′ C ATG GGT GGT GGA TTT GCT YFP
3′ G TAC CCA CCA CCT AAA .GA YFP
1. 3′ end, before annealing
                E N L Y F Q N L Y F Q G H H H H H H H H
5′ YFP G A AAT TTA TAC TTC CAA GGT CAT CAT CAC CAT CAT CAC CAT CAC
3′ YFP CTT TTA AAT ATG AAG GTT CCA GTA GTA GTG GTA GTA GTG GTA GTG
    H H X
CAT CAT TAA
GTA GTA ATT
2. 3′ end, after annealing
                E N L Y F Q G H H H H H H H H H H X
5′ YFP G.A AAT TTA TAC TTC CAA GGT CAT CAT CAC CAT CAT CAC CAT CAC CAT CAT TAA
3′ YFP CTT TTA AAT ATG AAG GTT CCA GTA GTA GTG GTA GTA GTG GTA GTG GTA GTA ATT
```

### BRIEF DESCRIPTION OF THE DRAWINGS

FIG. 1. Outline of the LIC-VBEx strategy. (a) A scheme illustrating the bisection of the L. lactis expression vector pNZxLIC. The segment containing the cat and LIC sequence is placed on an E. coli backbone, yielding pRExLIC. This vector can subsequently be used for the LIC procedure (depicted in b). Derivatives of pRExLIC containing inserts are converted to pNZxLIC derivatives by the VBEx procedure. (c) The SfiI sites flanking both segments of the plasmid vectors yield different overhangs. (d) Overexpression of membrane proteins in L. lactis. Left panels represent Coomassie stained protein gels, right panels immunoblots decorated with anti-His antibody. Minus and plus signs indicate samples uninduced and induced with nisin A. Filled and open arrows indicate His-tagged and non-tagged subunits/proteins, respectively. Marker bands are 170, 130, 100, 70, 55, 40, 35, 25, and 15 kDa. Shown are, from left to right: an ABC transporter and secondary riboflavin transporter from L. lactis, ABC transporters from Staphylococcus aureus and Streptococcus pneumoniae, and the integral membrane component of a TRAP transporter from Haemophilus influenzae; accession numbers are indicated below the panels.

FIG. 2. Analysis of SfiI characteristics. (a) The occurrence of SfiI sites in 492 prokaryotic genomes and the concatinated gene transcripts of 30 eukaryotes as a function of the GC-content of the DNA. As 64 different overhangs can be generated after SfiI digestion, the frequency of complementary SfiI sites is even lower (FIG. 3). (b) The number of SfiI overhangs not present in a genome or combined gene transcript as a function of the GC-content of the DNA (maximal 64). In rare cases where inserts would contain a SfiI site interfering with the VBEx procedure, non-occuring overhangs could be used to flank both segments of the vector.

FIG. 3. Analysis of the occurrence of SfiI sites yielding identical overhangs in genomes and combined transcripts as a function of the GC-content of the DNA. For each DNA, the datapoint of the most occurring SfiI site of this type is shown.

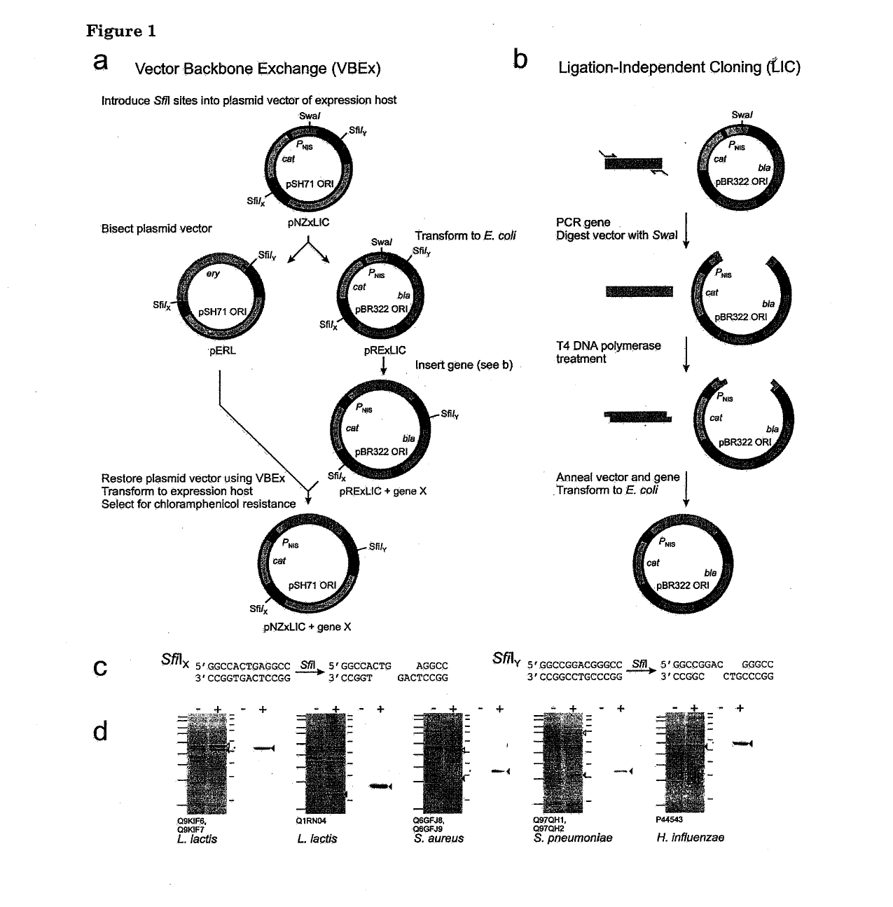

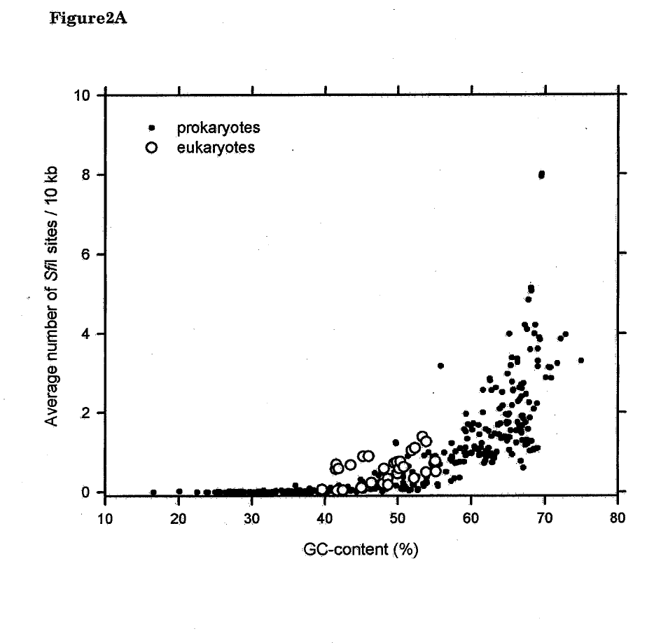

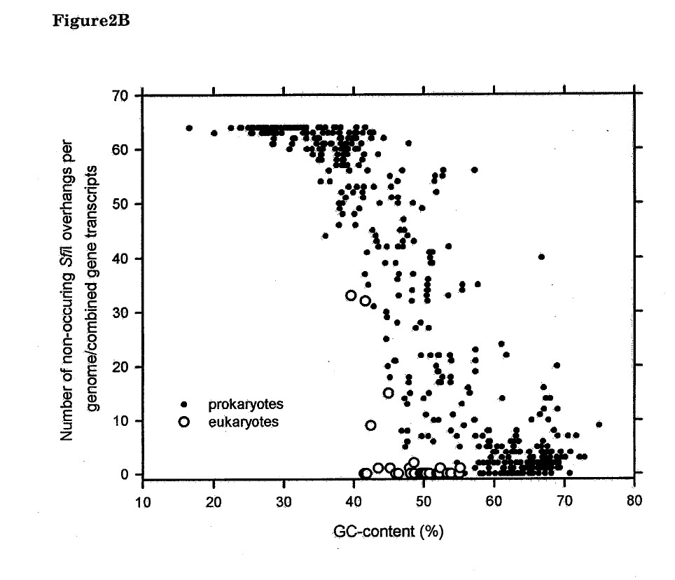

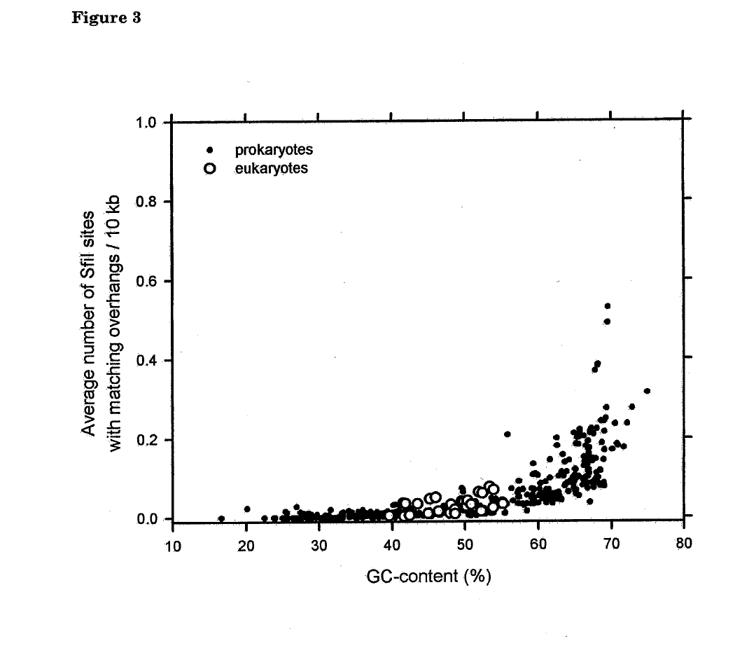

### REFERENCES

Aslanidis, C and P. J. de Jong, Nucleic Acids Res 18 (20), 6069 (1990).

Birnboim and Doly, Nucleic Acids Res. 1979 Nov. 24; 7(6):1513-1523

Bron P A, Benchimol M G, Lambert J, Palumbo E, Deghorain M, Delcour J, De Vos W M, Kleerebezem M, Hols P. Use of the alr gene as a food-grade selection marker in lactic acid bacteria. Appl Environ Microbiol. 2002 November; 68(11):5663-70

de Jong, R. N. and M. A. Daniels, R. Kaptein et al., J Struct Funct Genomics (2007).

de Ruyter, P. G. and O. P. Kuipers, and W. M. de Vos, Appl Environ Microbiol 62 (10), 3662 (1996).

El Demerdash H A, Heller K J, Geis A. Application of the shsp gene, encoding a small heat shock protein, as a food-grade selection marker for lactic acid bacteria. Appl Environ Microbiol. 2003 August; 69(8):4408-12.

Guzman, L. M. and D. Belin, M. J. Carson et al., J Bacteriol 177 (14), 4121 (1995).

Holo, H and I. F. Nes, Appl Environ Microbiol 55 (12), 3119 (1989).

Li, M. Z. and S. J. Elledge, Nat Methods 4 (3), 251 (2007).

Liu, Q and M. Z. Li, and D. Leibham et al., Curr Biol 8 (24), 1300 (1998).

Surade, S. and M. Klein, P. C. Stolt-Bergner et al., Protein Sci 15 (9), 2178 (2006)

Terzaghi, B. E. and W. E. Sandine, Appl Microbiol 29 (6), 807 (1975).

Walhout, A. J. and G. F. Temple and M. A. Brasch et al., Methods Enzymol 328, 575 (2000).

Wells, J. M. and P. W. Wilson and R. W. Le Page, J Appl Bacteriol 74 (6), 629 (1993).

TABLE 2
Available LIC vectors.

| | | Protein sequence after TEV | |
|---|---|---|---|
| Vector name | Protein sequence | protease cleavage | Expression host |
| pREnLIC | M-His₁₀-G-TEV site-protein | G-protein | L. lactis NZ9000 |
| pREcLIC | MGGGFA-protein-TEV site-His₁₀ | MGGGFA-protein-ENLYFQ | L. lactis NZ9000 |
| pREcLIC-GFP | MGGGFA-protein-TEV site-GFP-His₁₀ | MGGGFA-protein-ENLYFQ | L. lactis NZ9000 |
| pRE-USP45-nLIC | M-ssUSP45¹-His₁₀-G-TEV site-protein | G-protein | L. lactis NZ9000 |
| pBADnLIC | M-His₁₀-G-TEV site-protein | G-protein | E. coli |
| pBADcLIC | MGGGFA-protein-TEV site-His₁₀ | MGGGFA-protein-ENLYFQ | E. coli |
| pBADcLIC-GFP | MGGGFA-protein-TEV site-GFP-His₁₀ | MGGGFA-protein-ENLYFQ | E. coli |
| pBAD-OmpA-nLIC | M-ssOmpA²-His₁₀-G-TEV site-protein | G-protein | E. coli |

¹ssUSP45 indicates the signal sequence of the L. lactis USP45 protein. The pRE-USP45-nLIC vector is used if the N-terminus of the membrane protein of interest is predicted to be on the outside of the cytoplasmic membrane, or to replace the signal sequence of the protein of interest with the ssUSP45.
²ssOmpA indicates the signal sequence of the E. coli OmpA protein. The pBAD-OmpA-nLIC vector is used if the N-terminus of the membrane protein of interest is predicted to be on the outside of the cytoplasmic membrane, or to replace the signal sequence of the protein of interest with the ssOmpA.

TABLE 3
Recalcitrant micro-organisms in terms of gene
cloning (e.g. construction of gene libraries)

Lactococcus lactis
Lactobacillus sp. (e.g. bulgaricus, helveticus,
plantarum)
Sulfolobus solfataricus
Archaea in general (incl. haloarchaea and others)
Streptococcus sp. (e.g. thermophilus, pyogenes, mutans)
Leuconostoc sp.
Bacillus thermophilus, Bacillus caldotenax

1-26. (canceled)

27. A method for cloning a nucleic acid sequence of interest in a first kind of micro-organism, the method comprising:
inserting said nucleic acid of interest into a nucleic acid vehicle, which vehicle comprises an origin of replication of a second kind of micro-organism;
cloning said vehicle in a culture of said second kind of micro-organism;
isolating cloned vehicle comprising said nucleic acid sequence of interest;
substituting the vehicle's origin of replication of a second kind of micro-organism by an origin of replication of said first kind of micro-organism; and
cloning the resulting vehicle in a culture of said first kind of micro-organism.

28. A method according to claim 1, wherein said resulting vehicle is essentially devoid of elements derived from said second kind of micro-organism

29. A method according to claim 1, comprising:
providing a first nucleic acid vehicle which comprises an origin of replication of said second kind of micro-organism;
introducing a nucleic acid sequence of interest into said first vehicle;
providing a second nucleic acid vehicle which comprises an origin of replication of said first kind of micro-organism; and
substituting a part of said first vehicle, which part comprises said origin of replication of said second micro-organism but not said nucleic acid of interest, by a part of said second vehicle, said part comprising said origin of replication of said first microorganism.

30. A method according to claim 3, wherein said part of said first vehicle has different, non-palindromic overhangs that are not compatible with each other and that are compatible with the overhangs of said part of said second vehicle.

31. A method according to claim 3, wherein said parts are obtained by cleavage of said vehicles by SfiI.

32. A method according to claim 1, wherein said nucleic acid of interest is inserted into said first vehicle and cloned in a culture of said second kind of micro-organism using a method selected from the group consisting of a ligation independent cloning (LIC) procedure, Gateway, Univector Plasmid-fusion System (UPS) and LIC variants such as Enzyme-Free Cloning (EFC) and Sequence and Ligation Independent Cloning (SLIC).

33. A method according to claim 1, wherein said first micro-organism is a micro-organism other then Escherichia coli, preferably a recalcitrant micro-organism.

34. A method according to claim 1, further comprising allowing expression of said nucleic acid of interest by a culture of said first kind of micro-organism.

35. A method according to claim 1, further comprising obtaining an expression product of said nucleic acid sequence of interest.

36. A cloning assay wherein a nucleic acid of interest is cloned and expressed by a method according to claim 1.

37. A kit of parts comprising:
a first nucleic acid vehicle which comprises an origin of replication of a second kind of micro-organism; and
a second nucleic acid vehicle which comprises an origin of replication of a first kind of micro-organism;
wherein each vehicle comprises at least two recombinase sites and/or restriction enzyme cleavage sites and wherein said recombinase sites and/or restriction enzyme cleavage sites are preferably not present within said origins of replication.

38. A kit of parts according to claim 11, wherein each vehicle comprises at least two restriction enzyme cleavage sites which, upon cleaving, yield two different, non-palindromic overhangs that are not compatible with each other.

39. A kit of parts according to claim 11, wherein said first vehicle and said second vehicle comprise the same kind of restriction enzyme cleavage sites which, upon cleaving, yield two different, non-palindromic overhangs that are not compatible with each other.

40. A kit of parts according to claim 11, wherein said restriction enzyme cleavage sites are SfiI cleavage sites.

41. A kit of parts according to claim 11, wherein said second vehicle comprises at least part of an origin of replication of a micro-organism other then Escherichia coli, preferably an origin of replication of a recalcitrant micro-organism.
